(19) United States
(12) Patent Application Publication
(10) Pub. No.: US 2026/0066253 A1
(43) Pub. Date: Mar. 5, 2026

(54) MASS SPECTROMETRY METHOD, DISCRIMINATION MODEL CREATION METHOD, FIBER DISCRIMINATION METHOD, PROGRAM, INFORMATION PROCESSING APPARATUS, AND FIBER DISCRIMINATION SYSTEM

(71) Applicant: SHIMADZU CORPORATION, Kyoto (JP)

(72) Inventor: Yuzo YAMAZAKI, Kyoto (JP)

(73) Assignee: SHIMADZU CORPORATION, Kyoto (JP)

(21) Appl. No.: 19/305,316

(22) Filed: Aug. 20, 2025

(30) Foreign Application Priority Data
Aug. 28, 2024 (JP) 2024-146213

(51) Int. Cl. H01J 49/16; G01N 33/44

(52) U.S. Cl. CPC H01J 49/164; G01N 33/442

(57) ABSTRACT

A mass spectrometry method according to the present disclosure is a mass spectrometry method in a method for discriminating whether a polyethylene terephthalate fiber contained in a target fiber is a recycled fiber. The mass spectrometry method includes a step of preparing a sample and a step of measuring the sample using matrix-assisted laser desorption/ionization mass spectrometry. The step of preparing the sample has a step of dissolving the target fiber in a first solvent to prepare a solution, a step of adding tetrahydrofuran to the solution to separate the solution into a precipitate and a supernatant, a step of obtaining the supernatant, and a step of preparing a mixed solution containing the supernatant and a matrix on a sample plate.

### TECHNICAL FIELD

The present disclosure relates to a mass spectrometry method, a discrimination model creation method, a fiber discrimination method, a program, an information processing apparatus, and a fiber discrimination system, and more particularly, to improving the accuracy of discriminating whether polyethylene terephthalate fiber contained in a fiber is a recycled fiber.

### BACKGROUND ART

Polyethylene terephthalate (PET) is widely used in apparel as a general-purpose polymer for synthetic fibers. Recycled PET fibers, produced by recycling used PET bottles, are often used in apparel.

In the apparel market, there is a trend to add value to apparel by using recycled PET fibers, and there is a concern that labeling regarding the use of recycled PET fibers may be falsified. Therefore, a method is needed to discriminate whether an apparel item is made using recycled PET fibers.

As a method for discriminating whether a PET fiber is a recycled fiber derived from PET bottles, the “Guidelines for Ensuring the Reliability of Labeling of Designated Procurement Items, etc., 4.8. Commentary on Survey Methods for Products Containing Recycled Plastics (by Resin Type), Ministry of the Environment, March 2014 edition” (Non-Patent Literature 1) discloses a discrimination method that uses the amount of PET cyclic oligomers measured by liquid chromatography as an index. Furthermore, Wanderson Romao, et al., “Fingerprinting of bottle-grade poly (ethylene terephthalate) via matrix-assisted laser desorption/ionization mass spectrometry,” Polymer Degradation and Stability, Volume 95, Issue 4, 2010, Pages 666-671 (Non-Patent Literature 2) discloses a discrimination method based on mass spectrum data obtained using Matrix-Assisted Laser Desorption/Ionization Time-of-Flight Mass Spectrometry (MALDI-TOF MS).

### PRIOR ART DOCUMENTS

### Non-Patent Literature

[Non-Patent Literature 1] Guidelines for Ensuring the Reliability of Labeling of Designated Procurement Items, etc., 4.8. Commentary on Survey Methods for Products Containing Recycled Plastics (by Resin Type), Ministry of the Environment, March 2014 edition
[Non-Patent Literature 2] Wanderson Romao, Marcos F. Franco, Amadeu H. Iglesias, Gustavo B. Sanvido, Danilo A. Maretto, Fabio C. Gozzo, Ronei J. Poppi, Marcos N. Eberlin, Marco-Aurelio De Paoli, “Fingerprinting of bottle-grade poly (ethylene terephthalate) via matrix-assisted laser desorption/ionization mass spectrometry,” Polymer Degradation and Stability, Volume 95, Issue 4, 2010, Pages 666-671.

### SUMMARY OF INVENTION

### Problem to be Solved by the Invention

Analysis by liquid chromatography takes a longer measurement time compared to other analytical techniques. Therefore, the discrimination method using liquid chromatography disclosed in Non-Patent Literature 1 may be limited in the number of samples that can be discriminated per unit of time.

Measurement by matrix-assisted laser desorption/ionization mass spectrometry (MALDI-MS), such as MALDI-TOF MS, has a shorter measurement time compared to measurement using liquid chromatography. Therefore, a discrimination method using MALDI-MS can discriminate whether PET fibers contained in a sample are recycled fibers in a shorter time compared to a discrimination method using liquid chromatography. However, in data obtained by measuring a sample containing components other than the PET oligomers that serve as discrimination indices with MALDI-MS, the mass-to-charge ratio and its intensity of ions derived from PET oligomers may be affected by components other than the PET oligomers. When such data is used, the accuracy of fiber discrimination in a discrimination method using MALDI-MS may decrease.

The present disclosure has been made in view of such circumstances, and its object is to improve the accuracy of discrimination of PET fibers using MALDI-MS.

### Means for Solving the Problem

A mass spectrometry method according to one aspect of the present disclosure is a mass spectrometry method in a method for discriminating whether a PET fiber contained in a target fiber is a recycled fiber. The mass spectrometry method includes a step of preparing a sample and a step of measuring the sample using MALDI-MS, wherein the step of preparing the sample includes a step of dissolving the target fiber in a first solvent to prepare a solution, a step of adding tetrahydrofuran to the solution to separate the solution into a precipitate and a supernatant, a step of obtaining the supernatant, and a step of preparing a mixed solution containing the supernatant and a matrix on a sample plate.

A discrimination model creation method according to another aspect of the present disclosure is a discrimination model creation method for creating a discrimination model used to discriminate whether a PET fiber is a recycled fiber. The method includes a step of acquiring a plurality of reference mass spectrum data obtained by measuring a plurality of reference fibers with MALDI-MS, wherein each of the plurality of reference mass spectrum data is associated with recycling information indicating whether a PET fiber contained in the corresponding reference fiber is a recycled fiber, and further includes a step of acquiring a theoretical value of a mass-to-charge ratio of at least one PET oligomer, a step of extracting a signal intensity corresponding to the theoretical value from each of the plurality of reference mass spectrum data, a step of performing multivariate analysis using the recycling information and the signal intensity, and a step of creating a discrimination model using the result of the multivariate analysis.

### Effects of the Invention

According to the present disclosure, it is possible to improve the accuracy of discrimination of PET fibers using MALDI-MS.

### BRIEF DESCRIPTION OF THE DRAWINGS

FIG. 1 is a schematic diagram showing the configuration of a fiber discrimination system according to an embodiment.

FIG. 2 is a diagram for explaining the process of analytical pretreatment.

FIG. 3 is a flowchart showing a process for acquiring mass spectrum data of a fiber containing PET.

FIG. 4 is an example of mass spectrum data obtained by analyzing PET oligomers.

FIG. 5 is a diagram for explaining a process of extracting signal intensity from mass spectrum data.

FIG. 6 is a diagram for explaining a method for creating a discrimination model for discriminating whether PET is a recycled fiber.

FIG. 7 is a diagram for explaining data used in multivariate analysis to create a discrimination model.

FIG. 8 is a diagram showing an example of the results of multivariate analysis.

FIG. 9 is a flowchart for explaining a procedure for creating a discrimination model.

FIG. 10 is a diagram for explaining a method for discriminating whether a PET fiber contained in a target fiber is a recycled fiber using a discrimination model.

FIG. 11 is a flowchart for explaining a procedure for discriminating whether a PET fiber contained in a fiber is a recycled fiber using a discrimination model.

### EMBODIMENTS FOR CARRYING OUT THE INVENTION

Hereinafter, embodiments of the present disclosure will be described in detail with reference to the drawings. In the drawings, the same or corresponding parts are denoted by the same reference numerals, and a description thereof will not be repeated.

### [Configuration of Fiber Discrimination System]

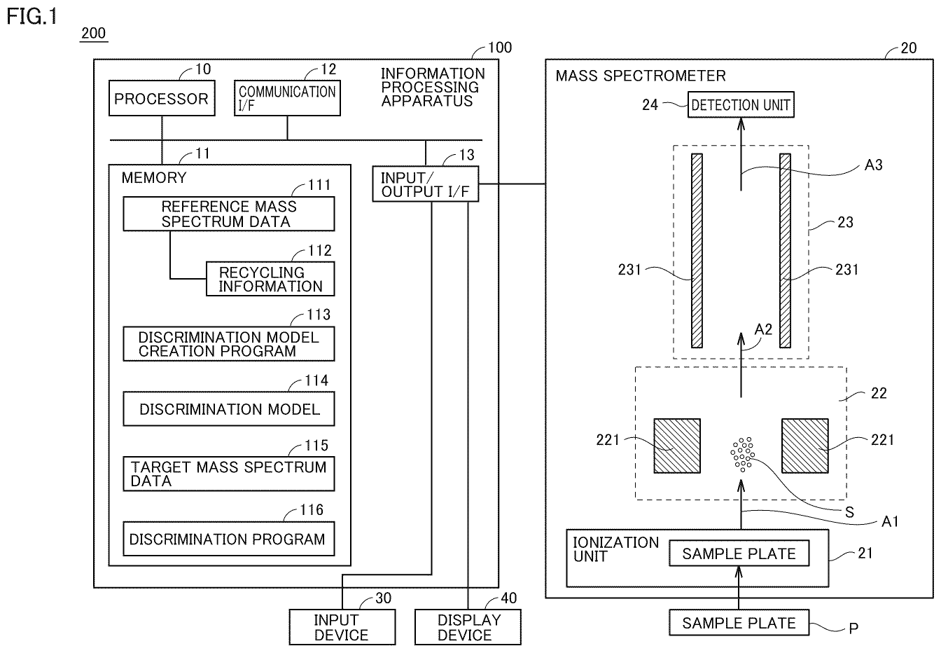

FIG. 1 is a diagram showing the configuration of a fiber discrimination system 200 according to an embodiment. Referring to FIG. 1, the fiber discrimination system 200 includes an information processing apparatus 100, a mass spectrometer 20, an input device 30, and a display device 40. The fiber discrimination system 200 discriminates whether a PET fiber contained in a target fiber (hereinafter referred to as a target fiber) is a recycled fiber based on mass spectrum data obtained by mass spectrometry of a sample prepared from the target fiber. The fiber discrimination system according to the present embodiment discriminates the target fiber based on the signal intensity of ions derived from PET oligomers in the mass spectrum data.

The mass spectrometer 20 is an apparatus for performing mass spectrometry on a sample. The analysis by the mass spectrometer 20 includes detecting peaks in the mass spectrum data and measuring the mass-to-charge ratio of substances contained in the sample. A mass spectrum is a plot with the mass-to-charge ratio on the horizontal axis and the signal intensity of the detected ions on the vertical axis. In this specification, a case where the mass spectrometer 20 is an apparatus that acquires mass spectrum data of a sample by Matrix-Assisted Laser Desorption/Ionization Time-of-Flight Mass Spectrometry (MALDI-TOF MS) will be described as an example, but the method of mass spectrometry is not particularly limited as long as it is possible to acquire appropriate ion intensity information regarding PET oligomer components.

The mass spectrometer 20 irradiates a laser onto a sample prepared on a sample plate P to ionize components in the sample. The mass spectrometer 20 separates and detects the ions according to the time it takes for the ions to fly through a flight tube. The flight time of the ions correlates with the mass-to-charge ratio of the component from which the ions originated.

The mass spectrometer 20 includes an ionization unit 21, an ion acceleration unit 22, a mass separation unit 23, and a detection unit 24. In FIG. 1, the movement of ions S in the mass spectrometer 20 is schematically shown by arrows A1, A2, and A3.

The ionization unit 21 ionizes substances in the sample by the MALDI method. The ionization unit 21 includes a sample plate holder (not shown) that supports the sample plate P, and an ion source including a laser device (not shown) that irradiates a laser beam onto the sample plate P. The type of laser device is not particularly limited as long as it can oscillate light that is absorbed by the selected matrix. The laser device is, for example, a nitrogen laser.

The sample plate P is a general MALDI plate, for example, a plate containing stainless steel or a conductive resin. Further, the sample plate P may be subjected to a surface treatment such as a water-repellent treatment or a hydrophilic treatment to improve sensitivity. A user drops a mixed solution of a specimen solution, a matrix, and a cationizing agent onto the sample plate P. When the solvent of the mixed solution evaporates, a mixed crystal of the specimen solution, the matrix, and the cationizing agent is formed on the sample plate P. The sample plate P with the mixed crystal formed on its surface is placed on a sample plate holder inside a vacuum chamber of the ionization unit 21. Note that the user may drop the mixed solution of the specimen solution, the matrix, and the cationizing agent onto the sample plate P, or may mix the specimen solution, the matrix, and the cationizing agent on the sample plate P to create a droplet of the mixed solution. In this specification, the above-mentioned mixed crystal corresponds to a sample for MALDI measurement.

The specimen solution is a supernatant obtained by removing impurities from a solution in which fibers containing PET have been dissolved, by a dissolution-reprecipitation method. A method for preparing a sample for MALDI measurement using this supernatant will be described later.

The matrix is not particularly limited as long as it can measure PET oligomers, but is, for example, Dithranol.

The cationizing agent is not particularly limited as long as it can promote the ionization of components by laser irradiation, but is, for example, sodium iodide.

The ionization unit 21, after reducing the pressure in the vacuum chamber where the sample plate P is placed, irradiates the mixed crystal on the sample plate P with a laser beam. Ions S generated in the ionization unit 21 by the laser beam irradiation are extracted by an electric field from an extraction electrode (not shown) and introduced into the ion acceleration unit 22, as indicated by arrow A1 in FIG. 1.

The ion acceleration unit 22 includes an acceleration electrode 221 and accelerates the introduced ions S. The flow of accelerated ions S is appropriately focused by an ion lens (not shown) and introduced into the mass separation unit 23, as indicated by arrow A2 in FIG. 1.

The mass separation unit 23 includes a flight tube 231 and separates the ions S based on the difference in flight time as each ion S flies through the interior of the flight tube 231. FIG. 1 shows an example of a linear type flight tube 231, but a reflectron type or multi-turn type flight tube can also be used. Note that the method of mass spectrometry is not particularly limited as long as it can separate and detect ions derived from compounds contained in the sample.

The detection unit 24 includes an ion detector, detects the ions S separated by the mass separation unit 23 as indicated by arrow A3, and outputs a detection signal with an intensity corresponding to the number of ions incident on the detection unit 24. The detection signal output from the detection unit 24 is input to the information processing apparatus 100.

In the fiber discrimination system 200, the mass spectrometer 20 performs mass spectrometry of PET oligomers in a sample prepared from fibers. That is, peaks corresponding to the mass-to-charge ratio of the PET oligomers in the sample are detected by mass spectrometry.

The mass spectrometer 20 analyzes the sample prepared on the sample plate P and generates mass spectrum data. The mass spectrometer 20 transmits the generated mass spectrum data to the information processing apparatus 100.

The information processing apparatus 100 includes, as main components, a processor 10, a memory 11, a communication interface (I/F) 12, and an input/output I/F 13. These units are communicably connected to each other via a bus. The information processing apparatus 100 is, for example, a computer. Note that the information processing apparatus 100 does not need to be configured by a single computer and may be configured by a plurality of computers.

The processor 10 is an example of an electric circuit and controls the operation of the information processing apparatus 100 by executing a given program. The program executed by the processor 10 may be stored in the memory 11 or in a storage device external to the information processing apparatus 100. The processor is, for example, a CPU (Central Processing Unit).

The memory 11 non-transitorily stores a program executed by the processor 10, mass spectrum data obtained by mass spectrometry, and a discrimination model. The program, mass spectrum data, and discrimination model stored in the memory 11 include reference mass spectrum data 111, recycling information 112, a discrimination model creation program 113, a discrimination model 114, target mass spectrum data 115, and a discrimination program 116. The memory 11 includes volatile memory (e.g., RAM (Random Access Memory)) and non-volatile memory (e.g., ROM (Read Only Memory), a hard disk drive, and a solid-state drive). Note that the above database and/or program may be stored in an external storage device accessible by the processor 10.

The reference mass spectrum data 111 is mass spectrum data obtained from fibers for which it is known whether the contained PET fibers are recycled fibers (hereinafter referred to as reference fibers). The recycling information 112 is information indicating whether the PET fibers contained in the reference fibers are recycled fibers, and is stored in the memory 11 in association with the corresponding reference mass spectrum data 111. The discrimination model creation program 113 is a program for creating a discrimination model 114 used for fiber discrimination, using the reference mass spectrum data 111 and the recycling information 112. A method for creating the discrimination model 114 will be described later.

The discrimination program 116 is a program that discriminates whether a PET fiber contained in a target fiber is a recycled fiber, using target mass spectrum data 115, which is mass spectrum data derived from the target fiber to be discriminated, and the discrimination model 114. Discrimination of the target fiber by the discrimination program 116 will be described later.

The communication I/F 12 is a communication interface for exchanging various data with external devices. The communication I/F 12 is realized by, for example, a network adapter. The communication method may be wireless communication such as Bluetooth (registered trademark) or wireless LAN, or wired communication using a USB (Universal Serial Bus) or the like.

The input/output I/F 13 is an interface for exchanging various data between the processor 10 and external devices connected to the input/output I/F 13. The external devices include the mass spectrometer 20, the input device 30, and the display device 40.

The information processing apparatus 100 may control the mass spectrometer 20, or another control device (for example, a computer) may be connected to the mass spectrometer 20, and the mass spectrometer 20 may be controlled by that control device.

The input device 30 includes, for example, at least one of a mouse, a keyboard, and a touch panel, and accepts operations for the information processing apparatus 100 and input of information to the information processing apparatus 100. This information is, for example, information regarding the recycling of reference fibers.

The display device 40 includes, for example, a liquid crystal display, an organic EL (Electro Luminescence) display, or the like, and displays information according to instructions from the information processing apparatus 100. This information is, for example, mass spectrum data and the discrimination result of the target fiber.

### Comparative Example

PET is widely used in apparel as a general-purpose polymer for synthetic fibers. Recycled PET fibers, produced by recycling used PET bottles, are often used in apparel.

In the apparel market, there is a trend to add value to apparel by using recycled PET fibers, and there is a concern that labeling regarding the use of recycled PET fibers may be falsified. Therefore, a method is needed to discriminate whether an apparel item is made using recycled PET fibers.

As a method for discriminating whether a PET fiber is a recycled fiber derived from PET bottles, Non-Patent Literature 1 discloses a discrimination method using the amount of PET cyclic oligomers obtained by measurement with a liquid chromatograph as an index. In the solid-state polymerization step performed in the process of manufacturing PET bottles, the amount of cyclic oligomers in PET decreases. Therefore, in the discrimination method using liquid chromatography, it is discriminated whether the PET fiber contained in the target fiber is a recycled fiber based on the measured value of the amount of PET cyclic oligomers in the fiber. However, the measurement time by liquid chromatography is longer than other analytical techniques, so the number of samples that can be measured per unit time is small. In addition, the amount of cyclic oligomers in the PET also decreases when the PET is dyed or subjected to hot water drawing treatment. Therefore, in the fiber discrimination method by liquid chromatography using the amount of PET cyclic oligomers as an index, the discrimination accuracy may decrease for fibers that have undergone dyeing and hot water drawing treatment.

Non-Patent Literature 2 discloses a discrimination method based on mass spectrum data obtained using MALDI-TOF MS. In the discrimination method using MALDI-TOF MS, fibers are discriminated based on the signal intensity of peaks derived from PET oligomers. Since MALDI-TOF MS requires a shorter measurement time than liquid chromatography, the number of samples that can be measured per unit time is greater than when using a liquid chromatograph. However, components other than oligomers contained in the sample may affect the measurement by MALDI-TOF MS, which may lead to a decrease in discrimination accuracy.

Focusing on the fact that PET used in PET bottles is copolymerized with isophthalic acid (IPA), a method has been proposed to discriminate whether a PET fiber is a recycled fiber derived from PET bottles by detecting a signal attributable to isophthalic acid IPA using FTIR or Raman spectroscopy. However, in this method, the accuracy of discrimination for blended fabrics mixed with fibers other than PET may decrease.

As described above, a discrimination method using MALDI-MS, such as MALDI-TOF MS, requires a shorter time for discrimination compared to other analytical techniques. On the other hand, there is a demand for improving the discrimination accuracy of this method.

### [Fiber Discrimination Method According to the Embodiment]

Therefore, the fiber discrimination method according to the present disclosure extracts signal intensities corresponding to PET oligomers from target mass spectrum data obtained by measuring a sample prepared by an analytical pretreatment using tetrahydrofuran (THF) with MALDI-MS, using the theoretical values of the mass-to-charge ratios of the oligomers as indices, and discriminates whether the PET fiber contained in the target fiber is a recycled fiber.

By using THE in the analytical pretreatment, components other than the PET oligomers that serve as discrimination indices in the discrimination using MALDI-MS (for example, polymers) can be removed. This can increase the degree of purification of the oligomers in the high molecular weight region that serve as discrimination indices in the discrimination using MALDI-MS. As a result, the S/N ratio of the signal corresponding to the oligomers in the high molecular weight region in the mass spectrum data of the target fiber can be improved, and the accuracy of fiber discrimination can be improved.

Furthermore, in the fiber discrimination method according to the present disclosure, the signal intensity corresponding to the PET oligomers is extracted from the mass spectrum data using the theoretical values of the mass-to-charge ratios of the PET oligomers that serve as discrimination indices. Therefore, it is not necessary to perform a process for identifying peaks or a process for correcting the mass-to-charge ratio in the obtained mass spectrum data. Furthermore, according to this discrimination method, it is possible to prevent a peak derived from a component other than the target component of the discrimination index from being erroneously used as a discrimination index. This makes it possible to accurately extract the signal intensity corresponding to the PET oligomers that serve as discrimination indices.

In the analytical pretreatment in the mass spectrometry method according to the present disclosure, it is not necessary to cause chemical reactions such as decomposing the PET polymer into monomers or esterifying the monomers. Therefore, the burden on the user for the analytical pretreatment can be reduced.

In addition, the fiber discrimination method according to the present disclosure discriminates fibers using data obtained by MALDI-MS. Therefore, the time required to acquire data is short compared to other analytical techniques. This can increase the number of samples that can be discriminated per unit of time.

Hereinafter, the specific processing content in the fiber discrimination method according to the embodiment will be described.

### [Analytical Pretreatment]

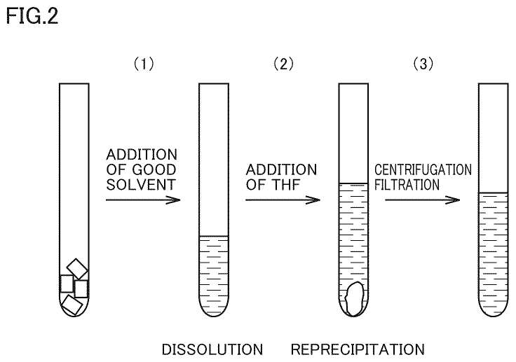

First, a mass spectrometry method for generating mass spectrum data will be described. FIG. 2 is a diagram for explaining an outline of the analytical pretreatment in the mass spectrometry method according to the present disclosure. In the analytical pretreatment for preparing a sample to be supplied to the mass spectrometer 20, first, (1) a first solvent is added to a container containing shredded fibers to dissolve the fibers. Next, (2) THF is added to the container. This causes the dissolved polymer molecules and impurities to precipitate. Then, (3) the solution after the addition of THF is centrifuged and filtered to remove the precipitate and obtain a supernatant. The supernatant obtained in (3) contains the PET oligomers that serve as discrimination indices in the discrimination method according to the present embodiment. The user prepares a sample using the supernatant obtained in (3) and measures it with the mass spectrometer 20.

Note that the type of the first solvent is not limited as long as it can dissolve the fibers to be subjected to mass spectrometry, but for example, a liquid in which hexafluoro-2-propanol (HFIP: Hexafluoroisopropylalcohol) and chloroform (Chloroform) are mixed at a volume ratio of 1:1 is used.

As for the liquid volume, for example, 100 mg of fibers are dissolved in 1 mL of the first solvent. 5 mL or more of THF is added to the solution.

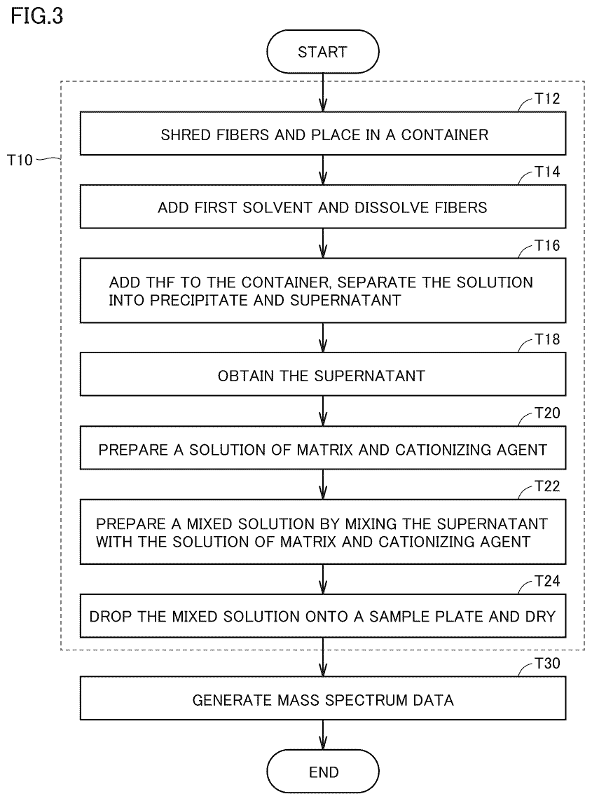

FIG. 3 is a flowchart showing the mass spectrometry process according to the embodiment. Among the steps shown in FIG. 3, step T10 is performed by a user's manual operation using experimental equipment used for general scientific experiments and mass spectrometry. Among the steps shown in FIG. 3, step T30 is executed by the mass spectrometer 20.

In step T10, the user prepares a sample to be supplied to the MALDI-MS. In FIG. 3, step T10 is shown subdivided as steps T12 to T24.

In step T12, the user shreds the fibers and puts them into a container.

In step T14, the user adds the first solvent to the container and dissolves the fibers. For 100 mg of fibers, for example, 1 mL of the first solvent is added.

In step T16, the user adds THF to the container and separates the solution into a precipitate and a supernatant. When the first solvent used in step T14 is 1 mL, for example, the user adds 5 mL or more of THF and adjusts the total liquid volume to 10 mL.

In step T18, the user removes the precipitate and obtains the supernatant.

In step T20, the user prepares a solution of a matrix and a cationizing agent. The matrix and the cationizing agent are dissolved in, for example, THF.

In step T22, the user prepares a mixed solution by mixing the supernatant obtained in step T16 and the solution of the matrix and cationizing agent prepared in step S18.

In step T24, the user drops the mixed solution prepared in step T22 onto the sample plate P and dries it.

In step T30, the sample plate P prepared in step T24 is supplied to the mass spectrometer 20, and the mass spectrometer 20 generates mass spectrum data of the sample.

According to the mass spectrometry method described above, components other than the PET oligomers that serve as discrimination indices in the discrimination using MALDI-MS are precipitated and removed. This can increase the degree of purification of the PET oligomers in the high molecular weight region in the supernatant. As a result, the S/N ratio of the peaks derived from the oligomers in the high molecular weight region in the mass spectrum data of the target fiber can be improved, and the accuracy of fiber discrimination can be improved.

### [Signal Intensity Extraction Method]

Next, a method for extracting the signal intensity corresponding to the PET oligomers used as discrimination indices from the mass spectrum data obtained by the above-described process will be described.

PET oligomers have four types of molecular structures: a linear oligomer with hydroxyl groups at both ends, a cyclic oligomer formed by the dehydration of one water molecule from both ends of the linear oligomer, a linear oligomers with a diethylene glycol linkage in the backbone with diethylene glycol (DEG) added to the end of the linear oligomer, and a cyclic oligomers with a diethylene glycol linkage in the backbone formed by the dehydration of one water molecule from both ends of the linear oligomers with a diethylene glycol linkage in the backbone.

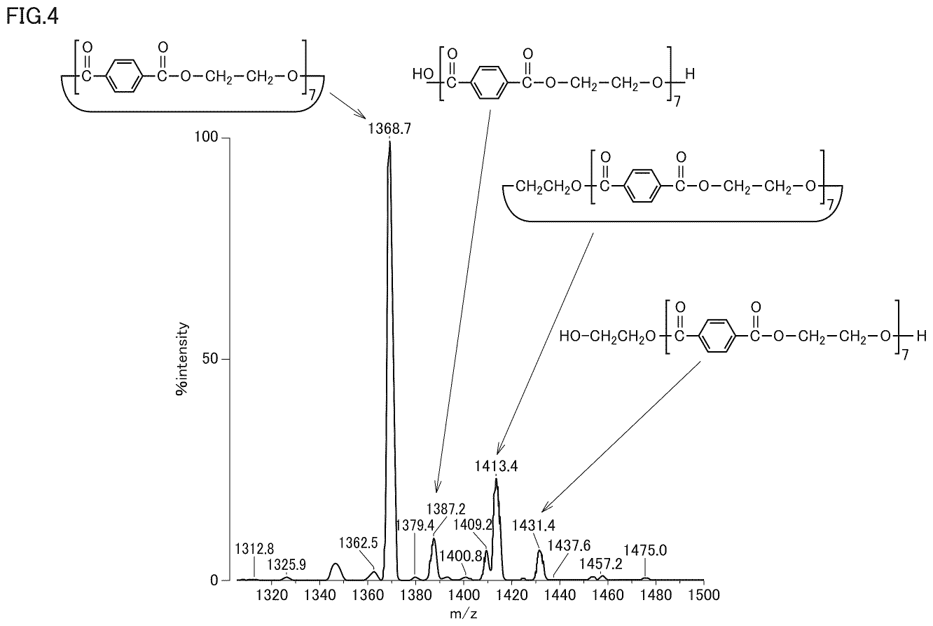

FIG. 4 shows an example of mass spectrum data acquired in the fiber discrimination method according to the present embodiment. In FIG. 4, the peak at m/z 1368.7 is derived from a 7-mer cyclic oligomer, and the peak at m/z 1387.2 is derived from a 7-mer linear oligomer. Also, the peak at m/z 1413.4 is derived from a 7-mer cyclic oligomers with a diethylene glycol linkage in the backbone, and the peak at m/z 1431.4 is derived from a 7-mer linear oligomers with a diethylene glycol linkage in the backbone.

Although FIG. 4 shows a mass-to-charge ratio range in which peaks derived from 7-mer molecules among PET oligomers are detected, the PET oligomers used as discrimination indices in the discrimination method according to the present embodiment are not limited to 7-mers. For example, oligomers from 3-mers to 10-mers may be used as discrimination indices. In this case, for each of the oligomers from 3-mers to 10-mers, since there are molecules having four types of molecular structures: linear oligomers, cyclic oligomers, linear oligomers with a diethylene glycol linkage in the backbones, and cyclic oligomers with a diethylene glycol linkage in the backbone, 8 (3- to 10-mers)×4 (4 types of molecular structures)=32 types of oligomers are used as discrimination indices.

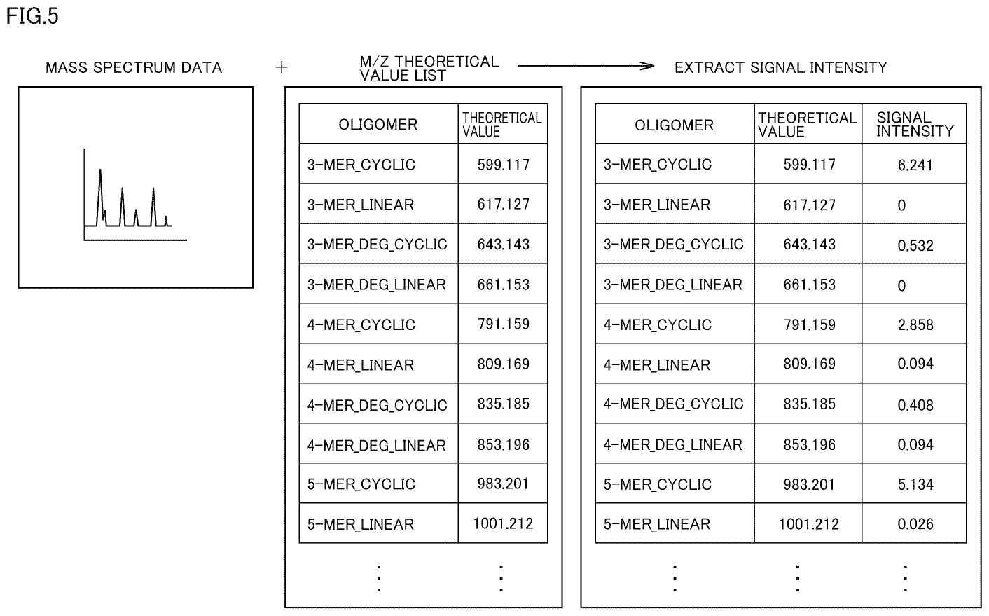

FIG. 5 is a diagram for explaining a method of extracting signal intensity by referring to the theoretical value of the mass-to-charge ratio of the PET oligomer used as a discrimination index from the obtained mass spectrum data.

As shown in FIG. 5, the information processing apparatus 100 calculates the theoretical values of the mass-to-charge ratios of ions derived from the PET oligomers to be used as discrimination indices and creates a list of the calculated theoretical values. Then, the information processing apparatus 100 extracts the signal intensity corresponding to the mass-to-charge ratio in the list from the mass spectrum data obtained by analyzing the fiber by mass spectrometry. Therefore, the information processing apparatus 100 can extract the signal intensity corresponding to the PET oligomers to be used as discrimination indices without performing a process of assigning peaks in the mass spectrum data.

Note that the PET oligomer to be used as a discrimination index is an oligomer of 1-mer or more and may be specified by the user or may be predetermined. The PET oligomers to be used as discrimination indices are, for example, 32 types of oligomers, which are 3- to 10-mer oligomers, with four types of molecular structures distinguished for each oligomer: linear oligomer, cyclic oligomer, linear oligomers with a diethylene glycol linkage in the backbone, and cyclic oligomers with a diethylene glycol linkage in the backbone.

Also, the theoretical value of the mass-to-charge ratio of the PET oligomer to be used as a discrimination index may be input by the user, or may be calculated by the information processing apparatus 100 based on the molecular formula of the oligomer.

In general, when a molecule is measured with a mass spectrometer 20, ions to which several types of cations are attached are detected. The mass-to-charge ratio of these ions differs depending on the attached cation. Therefore, several types of peaks derived from the same molecule with different types of attached cations may be detected in the mass spectrum data. In the fiber discrimination method according to the present embodiment, to prevent such a situation, the sample and the matrix are mixed with a cationizing agent and dropped onto the sample plate P. This can prevent the detection of ions to which multiple types of cations are attached. The cationizing agent is, for example, sodium iodide, and in this case, ions to which sodium ions are attached are detected. In this case, the theoretical value used for the mass-to-charge ratio of the PET oligomer as the discrimination index is the theoretical value for when a sodium ion is attached.

Further, in the signal intensity extraction method according to the present embodiment, it is preferable that there is one theoretical value corresponding to each oligomer. Therefore, it is preferable that the mass spectrum data is acquired in a linear mode in which ions fly linearly. The obtained mass spectrum data may be subjected to a smoothing process.

According to the signal intensity extraction method described above, the signal intensity is extracted from the mass spectrum data using the theoretical value of the mass-to-charge ratio of the PET oligomer to be used as a discrimination index. Therefore, it is not necessary to perform a process for identifying peaks or a process for correcting the mass-to-charge ratio in the obtained mass spectrum data. According to the signal intensity extraction method of the present embodiment, it is possible to prevent the extracted signal intensity value from being different from the obtained mass spectrum data.

Furthermore, according to the signal intensity extraction method described above, since the signal intensity corresponding to the theoretical value of the mass-to-charge ratio of the PET oligomer to be used as a discrimination index is extracted from the mass spectrum data, it is possible to prevent a peak derived from a component other than the PET oligomer (for example, other blended polymer fibers and surfactants mixed in during manufacturing) from being erroneously used as a discrimination index.

### [Creation of Discrimination Model]

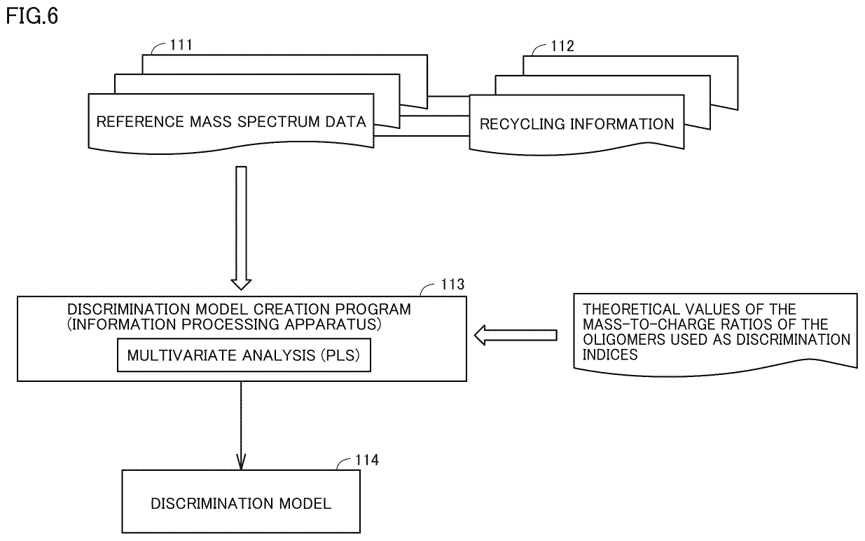

Next, a procedure for creating the discrimination model 114 will be described. FIG. 6 is a block diagram for explaining the procedure for creating the discrimination model 114.

As shown in FIG. 6, the information processing apparatus 100 executes the discrimination model creation program 113 to create the discrimination model 114. Specifically, it calls the reference mass spectrum data 111, which is mass spectrum data derived from reference fibers, from the memory 11. The reference mass spectrum data 111 is associated with recycling information 112 indicating whether the PET fiber contained in the corresponding reference fiber is a recycled fiber. The information processing apparatus 100 acquires the theoretical value of the mass-to-charge ratio of the PET oligomer to be used as a discrimination index. The information processing apparatus 100 extracts the signal intensity of the PET oligomer to be used as a discrimination index from the reference mass spectrum data 111 by the signal intensity extraction method described above.

The reference mass spectrum data 111 is mass spectrum data obtained by measuring a sample prepared from a reference fiber, for which it is known whether the contained PET fiber is a recycled fiber, with the mass spectrometer 20 by the mass spectrometry method described above. Specifically, the reference mass spectrum data 111 includes mass spectrum data of a sample prepared from a plurality of fibers containing recycled PET fibers and mass spectrum data of a sample prepared from a plurality of fibers not containing recycled PET fibers.

The recycling information 112 is information indicating whether the PET fiber contained in the reference fiber is a recycled fiber, and is stored in the memory 11 in association with the corresponding reference mass spectrum data 111.

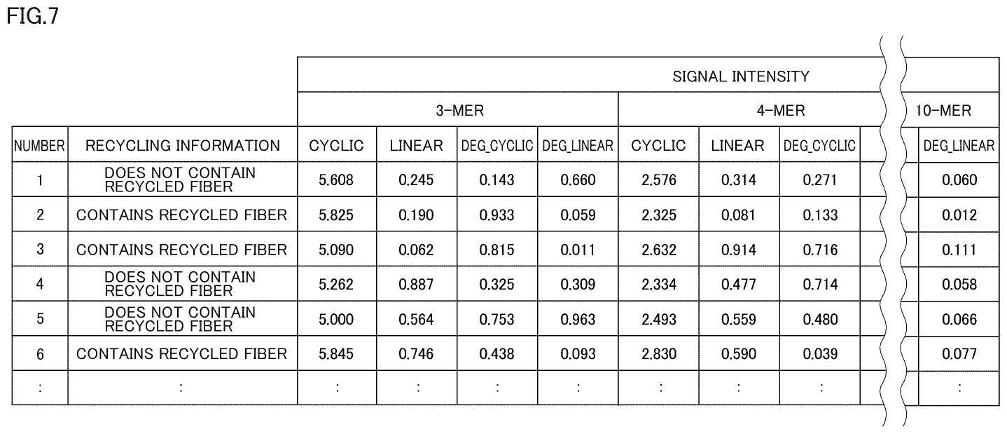

The information processing apparatus 100 performs multivariate analysis using the signal intensity information and the recycling information 112. FIG. 7 is an example of data used by the discrimination model creation program 113. In FIG. 7, “CYCLIC” indicates a cyclic oligomer, “LINEAR” indicates a linear oligomer, “DEG_CYCLIC” indicates a cyclic oligomers with a diethylene glycol linkage in the backbone, and “DEG LINEAR” indicates a linear oligomers with a diethylene glycol linkage in the backbone. As shown in FIG. 7, multivariate analysis is performed using data including recycling information and the signal intensities extracted from the reference mass spectrum data of the reference fiber corresponding to the recycling information.

The multivariate analysis is, for example, an analysis using the partial least squares (PLS) method. The information processing apparatus 100, by multivariate analysis, for example, for each of 8 types of oligomers (3- to 10-mer oligomers), distinguishes 4 types of molecular structures (linear oligomer, cyclic oligomer, linear oligomers with a diethylene glycol linkage in the backbone, and cyclic oligomers with a diethylene glycol linkage in the backbone), and uses the signal intensities of a total of 32 types of oligomers as variables to calculate factors and their loadings for classifying fibers containing recycled PET fibers from those that do not. Specifically, the signal intensities of the 32 types of oligomers mentioned above are used as explanatory variables in the analysis, and whether the PET fiber contained in the corresponding fiber is a recycled fiber is used as the objective variable in the analysis.

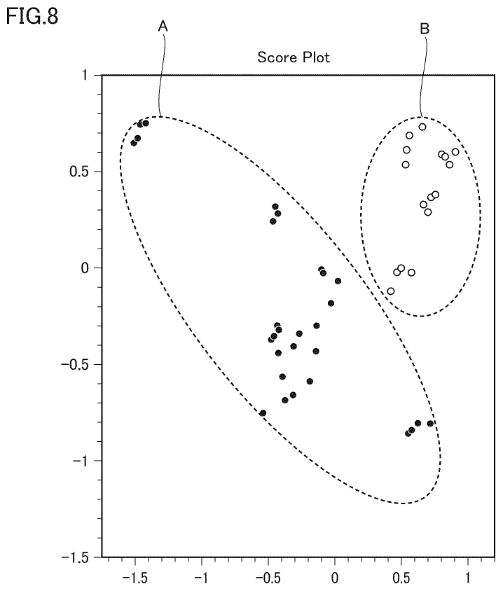

FIG. 8 shows a score plot by PLS as an example of the results of multivariate analysis. In FIG. 8, solid circles indicate data points obtained from fibers that do not contain recycled PET fibers, and open circles indicate data points obtained from recycled PET fibers.

As shown in FIG. 8, the data points obtained from fibers not containing recycled PET fibers are plotted in the range of region A, and the data points obtained from recycled PET fibers are plotted in the range of region B, and they are grouped according to whether the reference fiber contains recycled PET fiber.

In this way, by multivariate analysis, the types of PET oligomers that serve as factors for separating fibers that do not contain recycled PET fibers from those that do, and their loadings, are clarified.

The information processing apparatus 100 generates the discrimination model 114 using the results of the multivariate analysis. Generating the discrimination model 114 means, for example, calculating the centroid of each of region A and region B in FIG. 8. The centroid is the average value of the discriminant function values of each group obtained as a result of the multivariate analysis. Alternatively, it is to find a line segment that separates the solid circles and the open circles in FIG. 8. The method of fiber discrimination using the discrimination model 114 will be described later.

### [Processing Flow (Creation of Discrimination Model)]

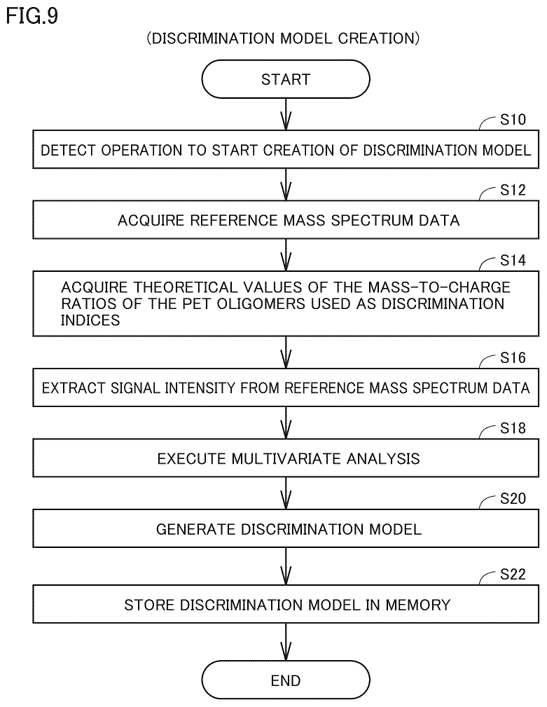

FIG. 9 is a flowchart for explaining the procedure for creating the discrimination model 114. The information processing apparatus 100 implements the processing of this flowchart by causing the processor 10 to execute the discrimination model creation program 113.

Referring to FIG. 9, in step S10, the processor 10 detects an operation to start the creation of the discrimination model 114. For example, when the user performs an operation to start the creation of the discrimination model 114 using the input device 30, that operation is detected in step S10.

In step S12, the processor 10 reads out the reference mass spectrum data 111 from the memory 11 and acquires it. The reference mass spectrum data 111 is associated with the recycling information 112.

In step S14, the processor 10 acquires the theoretical value of the mass-to-charge ratio of the PET oligomer to be used as a discrimination index. The oligomer to be used as a discrimination index may be specified by the user or may be predetermined. Also, the theoretical value of the mass-to-charge ratio of the oligomer to be used as a discrimination index may be input by the user, or may be calculated by the information processing apparatus 100 based on the molecular formula.

In step S16, the processor 10 extracts the signal intensity of the mass-to-charge ratio corresponding to the theoretical value of the oligomer to be used as a discrimination index from the reference mass spectrum data 111.

In step S18, the processor 10 executes multivariate analysis using the recycling information 112 acquired in step S12 and the signal intensity extracted in step S16.

In step S20, the processor 10 generates a discrimination model based on the result of the multivariate analysis in step S18.

In step S22, the processor 10 stores the discrimination model 114 generated in step S20 in the memory 11. Thereafter, the processor 10 ends the series of processes shown in FIG. 9.

### [Discrimination of Target Fiber]

A method for discriminating whether a PET fiber contained in a target fiber is a recycled fiber using the discrimination model 114 will be described.

The information processing apparatus 100 executes the discrimination program 116 to determine whether the PET fiber contained in the target fiber is a recycled fiber. Specifically, first, the target mass spectrum data 115, which is mass spectrum data derived from the target fiber, is called from the memory 11. The target mass spectrum data 115 is preferably mass spectrum data obtained from the target fiber to be discriminated by the mass spectrometry method described above.

Subsequently, the information processing apparatus 100 extracts the signal intensity of the oligomer to be used as a discrimination index from the target mass spectrum data 115 by the signal intensity extraction method described above. The oligomer to be used as a discrimination index is, for example, a 3- to 10-mer oligomer, and for each oligomer, 32 types of oligomers with four types of molecular structures distinguished: linear oligomer, cyclic oligomer, linear oligomers with a diethylene glycol linkage in the backbone, and cyclic oligomers with a diethylene glycol linkage in the backbone.

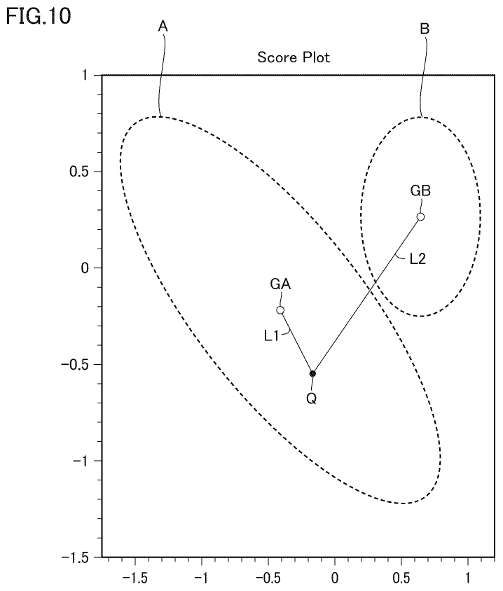

Then, the information processing apparatus 100 inputs the signal intensity of the oligomer, which is used as a discrimination index and extracted from the target mass spectrum data 115, into the discrimination model 114. FIG. 10 is a diagram for explaining a method of discriminating a fiber based on the signal intensity of the target mass spectrum data obtained from the target fiber.

FIG. 10 shows a score plot in the multivariate analysis performed in the process of creating the discrimination model 114. In FIG. 10, region A indicates a region where data points obtained from fibers not containing recycled PET fibers are plotted, and region B indicates a region where data points obtained from fibers containing recycled PET fibers are plotted. In FIG. 10, point GA indicates the centroid of region A, and point GB indicates the centroid of region B. A point Q is plotted on the score plot based on the signal intensity extracted from the target mass spectrum data 115 input from the information processing apparatus 100. The distances between the point Q derived from the target mass spectrum data 115 and the points GA and GB are shown by lines L1 and L2, respectively. In FIG. 10, since line L1<line L2, it is determined that point Q is closer to region A, which represents fibers that do not contain recycled PET fibers. Then, the discrimination program 116 outputs a discrimination result that the target fiber is a fiber that does not contain recycled PET fibers.

Note that, although FIG. 10 shows an example of a discrimination model in which the average value of the discriminant function values of each group obtained as a result of multivariate analysis is taken as the centroid, the distance from a point specified by the discriminant function value calculated from the signal intensity of the target fiber to the centroid of each group is calculated, and the target fiber is discriminated as belonging to the group of the centroid that is closer to the said point, the discrimination model 114 in the discrimination method according to the present embodiment is not limited to this. For example, the discrimination model 114 may discriminate the target fiber by linear discriminant analysis.

The information processing apparatus 100 outputs a display signal for displaying the obtained discrimination result to the display device 40. The user can recognize whether the PET fiber contained in the target fiber is a recycled fiber by checking the content displayed on the display device 40.

### [Processing Flow (Discrimination of Target Fiber)]

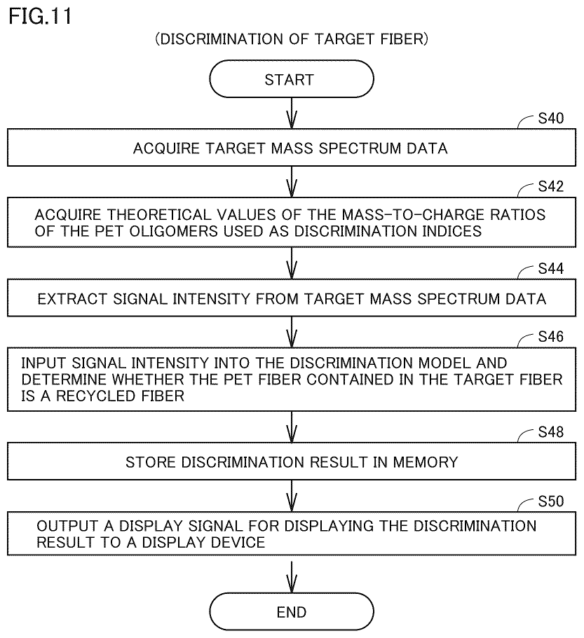

A procedure for discriminating whether a PET fiber contained in a target fiber is a recycled fiber will be described with reference to a flowchart. FIG. 11 is a flowchart for explaining a procedure for discriminating a target fiber based on target mass spectrum data 115 obtained from the target fiber, using the discrimination model 114. The processing of this flowchart is realized by the processor 10 of the information processing apparatus 100 executing the discrimination program 116.

Referring to FIG. 11, in step S40, the processor 10 reads out the target mass spectrum data 115 from the memory 11 and acquires it. The target mass spectrum data 115 is input to the memory 11 of the information processing apparatus 100 through a measuring instrument such as the mass spectrometer 20 connected to the input/output I/F 13. The data acquired in step S40 is mass spectrum data derived from the target fiber to be discriminated. Note that the target mass spectrum data 115 is preferably mass spectrum data acquired by the mass spectrometry method described above.

In step S42, the processor 10 acquires the theoretical value of the mass-to-charge ratio of the oligomer to be used as a discrimination index. The oligomer to be used as a discrimination index may be specified by the user or may be predetermined. Also, the theoretical value of the mass-to-charge ratio of the oligomer to be used as a discrimination index may be input by the user, or may be calculated by the information processing apparatus 100 based on the molecular formula.

In step S44, the processor 10 extracts the signal intensity of the mass-to-charge ratio corresponding to the theoretical value of the oligomer to be used as a discrimination index from the target mass spectrum data 115.

In step S46, the processor 10 inputs the signal intensity extracted in step S44 into the discrimination model 114 and determines whether the PET fiber contained in the target fiber is a recycled fiber.

In step S48, the processor 10 stores the discrimination result, which is the result of the determination in step S46, in the memory 11.

In step S50, the processor 10 outputs a display signal for displaying the discrimination result from step S46 to the display device 40. Thereafter, the processor 10 ends the series of processes shown in FIG. 11.

In the fiber discrimination method according to the present embodiment, THF is used in the analytical pretreatment. By using THE in the analytical pretreatment, components other than the oligomers that serve as discrimination indices in the discrimination using MALDI-MS (for example, polymers) can be removed. This can increase the degree of purification of the oligomers in the high molecular weight region that serve as discrimination indices in the discrimination using MALDI-MS. As a result, the S/N ratio of the peaks derived from the oligomers in the high molecular weight region in the mass spectrum data of the target fiber can be improved, and the accuracy of fiber discrimination can be improved.

Furthermore, in the fiber discrimination method according to the present disclosure, the signal intensity is extracted from the mass spectrum data using the theoretical value of the mass-to-charge ratio of the oligomer that serves as a discrimination index. Therefore, it is not necessary to perform a process for identifying peaks or a process for correcting the mass-to-charge ratio in the obtained mass spectrum data. Furthermore, there is no risk of erroneously using a peak derived from a component other than the target component of the discrimination index as a discrimination index. This makes it possible to accurately extract the signal intensity corresponding to the oligomer that serves as a discrimination index.

In the analytical pretreatment in the mass spectrometry method according to the present disclosure, it is not necessary to cause chemical reactions such as decomposing the PET polymer into monomers or esterifying the monomers. Therefore, the burden on the user for the analytical pretreatment can be reduced.

In addition, the fiber discrimination method according to the present disclosure uses data obtained by MALDI-MS. Therefore, the time required for measurement is short compared to other analytical techniques. This can increase the number of samples that can be discriminated per unit of time.

### [Aspects]

It will be understood by those skilled in the art that the embodiments and modifications thereof described above are specific examples of the following aspects.

(Item 1) A mass spectrometry method in a method for discriminating whether a polyethylene terephthalate fiber contained in a target fiber is a recycled fiber, the mass spectrometry method may comprise: a step of preparing a sample; and a step of measuring the sample using matrix-assisted laser desorption/ionization mass spectrometry (MALDI-MS), wherein the step of preparing the sample has: a step of dissolving the target fiber in a first solvent to prepare a solution; a step of adding tetrahydrofuran to the solution to separate the solution into a precipitate and a supernatant; a step of obtaining the supernatant; and a step of preparing a mixed solution containing the supernatant and a matrix on a sample plate.

According to the mass spectrometry method of Item 1, it is possible to improve the S/N ratio of peaks derived from oligomers in the high molecular weight region in the mass spectrum data of the fiber, and to improve the accuracy of fiber discrimination.

(Item 2) In the mass spectrometry method of Item 1, the first solvent may be a liquid in which hexafluoro-2-propanol and chloroform are mixed at a 1:1 volume ratio.

According to the mass spectrometry method of Item 2, molecules other than PET oligomers dissolved in a liquid mixture of hexafluoro-2-propanol and chloroform at a 1:1 volume ratio are precipitated by the addition of THF.

(Item 3) In the separating step in the mass spectrometry method of Item 1 or 2, 5 ml or more of the tetrahydrofuran may be added per 100 mg of the target fiber.

According to the mass spectrometry method of Item 3, it is possible to precipitate molecules other than oligomers while keeping the oligomers dissolved.

(Item 4) In the step of preparing the sample in the mass spectrometry method of any one of Items 1 to 3, 5 ml or more of the tetrahydrofuran may be added per 1 mL of the first solvent.

According to the mass spectrometry method of Item 4, it is possible to precipitate molecules other than oligomers while keeping the oligomers dissolved.

(Item 5) In the mass spectrometry method of any one of Items 1 to 4, the matrix may include dithranol.

According to the mass spectrometry method of Item 5, it is possible to improve the detection efficiency of PET oligomers in MALDI-MS.

(Item 6) In the mass spectrometry method of any one of Items 1 to 5, the mixed solution may include sodium iodide.

According to the mass spectrometry method of Item 6, when PET oligomers are ionized by laser irradiation, they become ions to which sodium ions are attached. As a result, the mass-to-charge ratio of the PET oligomers in the obtained mass spectrum data corresponds to the sodium-adduct ions of each oligomer.

(Item 7) In the mass spectrometry method of any one of Items 1 to 6, the MALDI-MS may be matrix-assisted laser desorption/ionization time-of-flight mass spectrometry.

According to the mass spectrometry method of Item 7, it is possible to perform fiber discrimination using mass spectrum data obtained by MALDI-TOF MS.

(Item 8) A discrimination model creation method for creating a discrimination model used to discriminate whether a polyethylene terephthalate (PET) fiber is a recycled fiber, the method may comprise: a step of acquiring a plurality of reference mass spectrum data obtained by measuring a plurality of reference fibers with MALDI-MS, wherein each of the plurality of reference mass spectrum data is associated with recycling information indicating whether a PET fiber contained in the corresponding reference fiber is a recycled fiber; and may further comprise a step of acquiring a theoretical value of a mass-to-charge ratio of at least one PET oligomer; a step of extracting a signal intensity corresponding to the theoretical value from each of the plurality of reference mass spectrum data; a step of performing multivariate analysis using the recycling information and the signal intensity; and a step of generating a discrimination model using the result of the multivariate analysis.

By using the discrimination model created by the discrimination model creation method of Item 8, it is possible to improve the accuracy of fiber discrimination.

(Item 9) In the discrimination model creation method of Item 8, the plurality of reference mass spectrum data may be generated by a mass spectrometry method having: a step of dissolving the reference fiber in a first solvent to prepare a solution; a step of adding tetrahydrofuran to the solution to separate the solution into a precipitate and a supernatant; a step of obtaining the supernatant; and a step of preparing a mixed solution containing the supernatant and a matrix on a sample plate.

According to the discrimination model creation method of Item 9, it is possible to improve the accuracy of fiber discrimination.

(Item 10) In the discrimination model creation method of any one of Item 8 or 9, the theoretical value may include the theoretical values of the mass-to-charge ratios of PET oligomers that are 3- to 10-mer oligomers and have four types of molecular structures: a linear oligomer with hydroxyl groups at both ends, a cyclic oligomer formed by the dehydration of one water molecule from both ends of the linear oligomer, a linear oligomers with a diethylene glycol linkage in the backbone with diethylene glycol (DEG) added to the end of the linear oligomer, and a cyclic oligomers with a diethylene glycol linkage in the backbone formed by the dehydration of one water molecule from both ends of the linear oligomers with a diethylene glycol linkage in the backbone.

According to the discrimination model creation method of Item 10, it is possible to create a discrimination model based on the signal intensities of PET oligomers that are 3- to 10-mer oligomers and have four types of molecular structures.

(Item 11) In the discrimination model creation method of any one of Items 8 to 10, the multivariate analysis may include the partial least squares method.

According to the discrimination model creation method of Item 11, it is possible to create a discrimination model using the partial least squares method.

(Item 12) In the discrimination model creation method of any one of Items 8 to 11, the MALDI-MS may be matrix-assisted laser desorption/ionization time-of-flight mass spectrometry.

According to the discrimination model creation method of Item 12, it is possible to create a discrimination model using mass spectrum data obtained by MALDI-TOF MS.

(Item 13) A program that, when executed by a processor installed in a computer, may cause the computer to execute the discrimination model creation method of any one of Items 8 to 12.

According to the program of Item 13, it is possible to improve the accuracy of fiber discrimination.

(Item 14) A fiber discrimination method for discriminating whether a polyethylene terephthalate (PET) fiber contained in a target fiber is a recycled fiber, the method may comprise: a step of acquiring target mass spectrum data obtained by measuring the target fiber with matrix-assisted laser desorption/ionization mass spectrometry; a step of acquiring a theoretical value of a mass-to-charge ratio of at least one PET oligomer; a step of extracting a signal intensity corresponding to the theoretical value from the target mass spectrum data; and a step of inputting the signal intensity to a discrimination model and determining whether the PET fiber contained in the target fiber is a recycled fiber, wherein the discrimination model may be created by the discrimination model creation method of any one of Items 8 to 12.

According to the fiber discrimination method of Item 14, it is possible to improve the accuracy of fiber discrimination.

(Item 15) In the fiber discrimination method of Item 14, each of the target mass spectrum data may be generated by a mass spectrometry method having: a step of dissolving the target fiber in a first solvent to prepare a solution; a step of adding tetrahydrofuran to the solution to separate the solution into a precipitate and a supernatant; a step of obtaining the supernatant; and a step of preparing a mixed solution containing the supernatant and a matrix on a sample plate.

According to the fiber discrimination method of Item 15, it is possible to improve the accuracy of fiber discrimination.

(Item 16) In the fiber discrimination method of Item 14 or 15, the theoretical value may include the theoretical values of the mass-to-charge ratios of PET oligomers that are 3- to 10-mer oligomers and have four types of molecular structures: a linear oligomer with hydroxyl groups at both ends, a cyclic oligomer formed by the dehydration of one water molecule from both ends of the linear oligomer, a linear oligomers with a diethylene glycol linkage in the backbone with diethylene glycol (DEG) added to the end of the linear oligomer, and a cyclic oligomers with a diethylene glycol linkage in the backbone formed by the dehydration of one water molecule from both ends of the linear oligomers with a diethylene glycol linkage in the backbone.

According to the fiber discrimination method of Item 16, it is possible to perform fiber discrimination based on the signal intensities of PET oligomers that are 3- to 10-mer oligomers and have four types of molecular structures.

(Item 17) The fiber discrimination method of any one of Items 14 to 16 may further comprise a step of displaying the discrimination result in the determining step.

According to the fiber discrimination method of Item 17, the user can easily recognize whether the PET fiber contained in the target fiber is a recycled fiber by checking the displayed discrimination result.

(Item 18) A program that, when executed by a processor installed in a computer, causes the computer to execute the fiber discrimination method of any one of Items 14 to 17.

According to the program of Item 18, it is possible to improve the accuracy of fiber discrimination.

(Item 19) An information processing apparatus may comprise: at least one or more processors; and a memory accessible by the one or more processors, wherein the memory stores one or more instructions to be executed by the processor, and the processor, by executing the one or more instructions: acquires target mass spectrum data obtained by measuring a target fiber with matrix-assisted laser desorption/ionization mass spectrometry; acquires a theoretical value of a mass-to-charge ratio of at least one PET oligomer; extracts a signal intensity corresponding to the theoretical value from the target mass spectrum data; inputs the signal intensity to a discrimination model and determines whether the PET fiber contained in the target fiber is a recycled fiber; and the discrimination model may be created by the discrimination model creation method of any one of Items 8 to 12.

According to the information processing apparatus of Item 19, it is possible to improve the accuracy of fiber discrimination.

(Item 20) A fiber discrimination system may comprise the information processing apparatus of Item 19 and a matrix-assisted laser desorption/ionization time-of-flight mass spectrometer.

According to the fiber discrimination system of Item 20, it is possible to discriminate whether a PET fiber contained in a target fiber is a recycled fiber using mass spectrum data obtained by MALDI-TOF MS.

It should be understood that the embodiments disclosed herein are illustrative and not restrictive in all respects. The scope of the present invention is indicated by the claims rather than by the foregoing description, and all changes that come within the meaning and range of equivalency of the claims are intended to be embraced therein.

### REFERENCE SIGNS LIST

10 processor, 11 memory, 12 communication interface, 20 mass spectrometer, 21 ionization unit, 22 ion acceleration unit, 23 mass separation unit, 24 detection unit, 30 input device, 40 display device, 100 information processing apparatus, 111 reference mass spectrum data, 112 recycling information, 113 discrimination model creation program, 114 discrimination model, 115 target mass spectrum data, 116 discrimination program, 200 fiber discrimination system, 221 acceleration electrode, 231 flight tube.

1. A mass spectrometry method in a method for discriminating whether a polyethylene terephthalate fiber contained in a target fiber is a recycled fiber, the mass spectrometry method comprising:
a step of preparing a sample; and
a step of measuring the sample using matrix-assisted laser desorption/ionization mass spectrometry (MALDI-MS),
wherein the step of preparing the sample comprises:
a step of dissolving the target fiber in a first solvent to prepare a solution;
a step of adding tetrahydrofuran to the solution to separate the solution into a precipitate and a supernatant;
a step of obtaining the supernatant; and
a step of preparing a mixed solution containing the supernatant and a matrix on a sample plate.

2. The mass spectrometry method according to claim 1, wherein the first solvent is a liquid in which hexafluoro-2-propanol and chloroform are mixed at a 1:1 volume ratio.

3. The mass spectrometry method according to claim 1, wherein, in the separating step, 5 ml or more of the tetrahydrofuran is added per 100 mg of the target fiber.

4. The mass spectrometry method according to claim 1, wherein, in the step of preparing the sample, 5 ml or more of the tetrahydrofuran is added per 1 mL of the first solvent.

5. The mass spectrometry method according to claim 1, wherein the matrix includes dithranol.

6. The mass spectrometry method according to claim 1, wherein the mixed solution includes sodium iodide.

7. The mass spectrometry method according to claim 1, wherein the MALDI-MS is matrix-assisted laser desorption/ionization time-of-flight mass spectrometry.

8. A discrimination model creation method for creating a discrimination model used to discriminate whether a polyethylene terephthalate (PET) fiber is a recycled fiber, the method comprising:
a step of acquiring a plurality of reference mass spectrum data obtained by measuring a plurality of reference fibers with MALDI-MS,
wherein each of the plurality of reference mass spectrum data is associated with recycling information indicating whether a PET fiber contained in the corresponding reference fiber is a recycled fiber,
the method further comprising:
a step of acquiring a theoretical value of a mass-to-charge ratio of at least one PET oligomer;
a step of extracting a signal intensity corresponding to the theoretical value from each of the plurality of reference mass spectrum data;
a step of performing multivariate analysis using the recycling information and the signal intensity; and
a step of generating a discrimination model using the result of the multivariate analysis.

9. The discrimination model creation method according to claim 8, wherein the plurality of reference mass spectrum data are generated by a mass spectrometry method comprising:
a step of dissolving the reference fiber in a first solvent to prepare a solution;
a step of adding tetrahydrofuran to the solution to separate the solution into a precipitate and a supernatant;
a step of obtaining the supernatant; and
a step of preparing a mixed solution containing the supernatant and a matrix on a sample plate.

10. The discrimination model creation method according to claim 8, wherein the theoretical value includes the theoretical values of the mass-to-charge ratios of PET oligomers that are 3- to 10-mer oligomers and have four types of molecular structures: a linear oligomer with hydroxyl groups at both ends, a cyclic oligomer formed by the dehydration of one water molecule from both ends of the linear oligomer, a linear oligomers with a diethylene glycol linkage in the backbone with diethylene glycol (DEG) added to the end of the linear oligomer, and a cyclic oligomers with a diethylene glycol linkage in the backbone formed by the dehydration of one water molecule from both ends of the linear oligomers with a diethylene glycol linkage in the backbone.

11. The discrimination model creation method according to claim 8, wherein the multivariate analysis includes a partial least squares method.

12. The discrimination model creation method according to claim 8, wherein the MALDI-MS is matrix-assisted laser desorption/ionization time-of-flight mass spectrometry.

13. A non-transitory computer-readable recording medium storing a program that, when executed by a processor installed in a computer, causes the computer to execute the discrimination model creation method according to claim 8.

14. A fiber discrimination method for discriminating whether a polyethylene terephthalate (PET) fiber contained in a target fiber is a recycled fiber, the method comprising:
a step of acquiring target mass spectrum data obtained by measuring the target fiber with matrix-assisted laser desorption/ionization mass spectrometry;
a step of acquiring a theoretical value of a mass-to-charge ratio of at least one PET oligomer;
a step of extracting a signal intensity corresponding to the theoretical value from the target mass spectrum data; and
a step of inputting the signal intensity to a discrimination model and determining whether the PET fiber contained in the target fiber is a recycled fiber,
wherein the discrimination model is created by the discrimination model creation method according to claim 8.

15. The fiber discrimination method according to claim 14, wherein each of the target mass spectrum data is generated by a mass spectrometry method comprising:
a step of dissolving the target fiber in a first solvent to prepare a solution;
a step of adding tetrahydrofuran to the solution to separate the solution into a precipitate and a supernatant;
a step of obtaining the supernatant; and
a step of preparing a mixed solution containing the supernatant and a matrix on a sample plate.

16. The fiber discrimination method according to claim 14, wherein the theoretical value includes the theoretical values of the mass-to-charge ratios of PET oligomers that are 3- to 10-mer oligomers and have four types of molecular structures: a linear oligomer with hydroxyl groups at both ends, a cyclic oligomer formed by the dehydration of one water molecule from both ends of the linear oligomer, a linear oligomers with a diethylene glycol linkage in the backbone with diethylene glycol (DEG) added to the end of the linear oligomer, and a cyclic oligomers with a diethylene glycol linkage in the backbone formed by the dehydration of one water molecule from both ends of the linear oligomers with a diethylene glycol linkage in the backbone.

17. The fiber discrimination method according to claim 14, further comprising a step of displaying a discrimination result in the determining step.

18. A non-transitory computer-readable recording medium storing a program that, when executed by a processor installed in a computer, causes the computer to execute the fiber discrimination method according to claim 14.

19. An information processing apparatus, comprising:
at least one or more processors; and
a memory accessible by the one or more processors,
wherein the memory stores one or more instructions to be executed by the processor, and the processor, by executing the one or more instructions:
acquires target mass spectrum data obtained by measuring a target fiber with matrix-assisted laser desorption/ionization mass spectrometry;
acquires a theoretical value of a mass-to-charge ratio of at least one PET oligomer;
extracts a signal intensity corresponding to the theoretical value from the target mass spectrum data;
inputs the signal intensity to a discrimination model and determines whether the PET fiber contained in the target fiber is a recycled fiber; and
the discrimination model is created by the discrimination model creation method according to claim 8.

20. A fiber discrimination system, comprising:
the information processing apparatus according to claim 19; and
a matrix-assisted laser desorption/ionization time-of-flight mass spectrometer.
